(19) United States
(12) Patent Application Publication
(10) Pub. No.: US 2013/0143812 A1
(43) Pub. Date: Jun. 6, 2013

(54) ANTI-INFLAMMATORY DRUG

(71) Applicants: Takashi Kadowaki, Tokyo (JP); Kohjiro Ueki, Tokyo (JP); Yukiko Okazaki, Tokyo (JP); Matthias Bluher, Leipzig (DE); Sumiko Ozawa, Tokyo (JP)

(72) Inventors: Takashi Kadowaki, Tokyo (JP); Kohjiro Ueki, Tokyo (JP); Yukiko Okazaki, Tokyo (JP); Matthias Bluher, Leipzig (DE); Sumiko Ozawa, Tokyo (JP)

(73) Assignees: SEKISUI MEDICAL CO., LTD., Tokyo (JP); THE UNIVERSITY OF TOKYO, Tokyo (JP)

(21) Appl. No.: 13/699,839

(22) PCT Filed: May 27, 2011
(86) PCT No.: PCT/JP2011/062162

§ 371 Date: Feb. 8, 2013

(30) Foreign Application Priority Data
May 27, 2010 (JP) 2010-122037

(51) Int. Cl. C07K 14/495

(52) U.S. Cl. CPC C07K 14/495
USPC 514/8.9; 530/399; 435/320.1

(57) ABSTRACT

Provided is a new anti-inflammatory drug that produces an anti-inflammatory effect by modulating macrophage function. Specifically, a new anti-inflammatory drug that produces an anti-inflammatory effect through induction of M2 macrophages using activin species is provided.

### TECHNICAL FIELD

The present invention relates to new anti-inflammatory drugs producing an anti-inflammatory effect by modulating macrophage function. Specifically, the present invention relates to new anti-inflammatory drugs producing an anti-inflammatory effect through the induction of M2 macrophages.

### BACKGROUND ART

Obesity causes a chronic low-level systemic inflammatory state. This is a basic clinical condition of metabolic disorders such as insulin resistance and type 2 diabetes. Among cells involved with an inflammation-promoting effect in obesity, the action of macrophages infiltrating in fat tissues is recently attracting attention.

From recent studies, it is believed that although inactive M2 macrophages produce IL-10, which is an anti-inflammatory cytokine, and arginase which suppresses NO biosynthesis to suppress inflammatory changes in non-obese visceral fat tissues, if active M1 macrophages increase due to obesity, inflammatory cytokines such as TNF-α and IL-6 are secreted, promoting inflammatory changes in fat tissues.

It is known that M1 macrophages are almost unrecognizable in non-obese visceral fat tissues while the number of infiltrating M1 macrophages increases due to obesity. M1 macrophages in fat tissues strongly express inflammatory cytokines such as TNF-α, IL-6, and MCP-1, and oxidative stress-related genes such as iNOS. Therefore, it is believed that M1 macrophages promote chronic inflammation and oxidative stress of visceral fat tissues and play an important role in the onset of insulin resistance associated with obesity.

In non-obese fat tissues, M2 macrophages are diffusely present. The number of M2 macrophages does not increase due to obesity. Although few reports are made on the effect of M2 macrophages on insulin sensitivity, it is inferred that M2 macrophages are involved with maintenance/improvement of insulin sensitivity. It is reported that M2 macrophages strongly express genes different from M1 macrophages, such as IL-10, arginase-1, Mrc1, YM1, and CD209 and that one of anti-inflammatory cytokines, IL-10, enhances insulin signaling in cultured adipocytes (Non-Patent Literature 1). Arginase strongly expressed in M2 macrophages acts competitively to iNOS. Since the oxidative stress of obese fat tissues promotes insulin resistance, it is thought that M2 macrophages highly expressing arginase are likely to be involved with improvement of insulin resistance.

Recently, it is gradually revealed that macrophage infiltration in fat tissues is induced by an increase in production of MCP-1 (monocyte chemoattractant protein-1, sometimes referred to as MCP1 in this description) in obese fat tissues. In mice systemically lacking MCP-1 or CCR2 (C—C Chemokine receptor 2), which is a major receptor for MCP-1, macrophages infiltrating in fat tissues are reduced and the reduction in TNF-α production and the improvement in systemic insulin resistance are reported in obesity due to high-fat diet feeding (Non-Patent Literatures 2 and 3). Conversely, in mice overexpressing MCP-1 specifically in the fat tissues, the macrophage infiltration and the increase in TNF-α production were observed in fat tissues and the systemic insulin resistance was deteriorated (Non-Patent Literatures 3 and 4).

Mice (db/WT:MCP-1⁻/WT) acquired from crossbreeding between genetically-obese db/db mice and MCP-1 knockout mice are reported to exhibit macrophage infiltration and insulin resistance at the same level as db/db mice, which indicates the involvement of a signal other than MCP-1 (Non-Patent Literature 5). Osteopontin or CXCL14 knockout mice or α4 integrin mutant mice are reported to exhibit the suppression of macrophage infiltration into fat tissues and the improvement in insulin resistance in high-fat diet-induced obesity (Non-Patent Literatures 6, 7, and 8).

Fat tissues produce a large number of inflammatory cytokines and anti-inflammatory cytokines and it is believed that a breakdown of balance between the both cytokines during increase in body fat amount is involved with the onset/development of the metabolic syndrome based on obesity. As described above, M1 macrophages and M2 macrophages are the main producer cells of inflammatory cytokines and anti-inflammatory cytokines, respectively, and it is conceivable that adjustment of the numbers and functions of the both macrophages possibly enables the suppression of inflammatory changes of adipocytes and the maintenance and control of homeostasis of systemic glycolipid metabolism.

The inventors have discovered that expression of follistatin-like protein 3 (FSTL3) increases in genetically-obese db/db mice and high-fat diet-fed mice or in adipocytes of human individuals having a tendency toward obesity (unpublished Japanese Patent Application No. 2010-122148). FSTL3 is a protein known to bind to activins to inhibit binding of activins to their receptor.

Although activins are cytokines belonging to the TGF-β super family and have a dimer structure as is the case with TGF-β etc., five types of sub-units (called β-subunits) making up the dimer are known, and it has been revealed that, in the case of mammals, βC and βE genes exist in addition to βA and βB (Non-Patent Literatures 9 and 10). Three types of activin species, i.e., activin A (βA-βA), activin AB (βA-βB), and activin B (βB-βB) have been identified as those expressed in vivo as proteins in mammals. Two types of genes, i.e., inhibin βA and βB genes code activin; a dimer formed from products of the inhibin βA gene is called activin A; a dimer formed from products of the inhibin βB gene is called activin B; and a dimer formed from respective products of the inhibin βA gene and the inhibin βB gene is called activin AB. It is thought that activin species (activin A, activin AB, or activin B) bind to an activin type II receptor (ActRIIA or ActRIIB) and that a complex thereof activates an activin type I receptor (ALK4) to transmit a signal (Non-Patent Literature 11).

Since both βA and βB of activin Are expressed in pancreas (Non-Patent Literatures 12, 13, and 14) and is thought to be important for functional differentiation of pancreatic β-cells (Non-Patent Literature 15), an activin has attracted attention as a therapeutic agent having a new pancreatic function-improvement effect in terms of a pancreas regenerating factor, and therefore, an antidiabetic drug has been proposed that contains an activin As an active ingredient (Patent Literature 1). However, no report has been made on activin species in terms of effect on macrophages and relation with inflammation.

### CITATION LIST

### Patent Literature

Patent Literature 1: Japanese Laid-Open Patent Publication No. 2003-113111

### Non-Patent Literatures

Non-Patent Literature 1: J. Clin. Invest., 117: 175-184, 2007
Non-Patent Literature 2: J. Clin. Invest., 116: 1494-1505, 2006
Non-Patent Literature 3: J. Clin. Invest., 116: 115-124, 2006
Non-Patent Literature 4: J. Bio. Chem., 281: 26602-26614, 2006
Non-Patent Literature 5: Diabetologia, 50: 471-480, 2007
Non-Patent Literature 6: J. Clin. Invest., 117: 2877-2888, 2007
Non-Patent Literature 7: J. Bio. Chem., 282: 30794-30803, 2007
Non-Patent Literature 8: Diabetes, 57: 1842-1851, 2008
Non-Patent Literature 9: Biochem. Biophys. Res. Commun., 228: 669-674, 1996
Non-Patent Literature 10: Biochim. Biophys. Acta., 1307: 145-148, 1996
Non-Patent Literature 11: Endocrinol., 141: 2281-2284, 2000
Non-Patent Literature 12: Endocrinology, 133: 624-630, 1993
Non-Patent Literature 13: FEBS Letters, 319: 217-220, 1993
Non-Patent Literature 14: J. Gastrointest. Surg., 4: 269-275, 2000
Non-Patent Literature 15: J. Clin. Invest., 102: 294-301, 1998

### SUMMARY OF INVENTION

### Technical Problem

It is an object of the present invention to provide a new anti-inflammatory drug producing an anti-inflammatory effect by modulating macrophage function. It is also an object of the present invention to provide a substance effectively suppressing inflammatory changes in adipocytes and a screening method for such substance.

### Solution to Problem

As a result of extensive research for solving the problems, the inventors have discovered that activin species (activin A, activin AB, or activin B) induces M2 macrophages and effectively suppresses inflammatory changes in adipocytes, thereby completing the present invention. The present invention provides the following.

(1) An anti-inflammatory drug containing an activin species as an active ingredient.

(2) The anti-inflammatory drug of (1), wherein said activin species is one or more species selected from activin A, activin AB, or activin B.

(3) The anti-inflammatory drug of (1) or (2), wherein said activin species is a vector in which a gene of said activin species is incorporated.

(4) An anti-inflammatory drug containing an inducer of M2 macrophages as an active ingredient.

(5) A method of treating or improving inflammation comprising: a step of administering an activin species to a subject.

### BRIEF DESCRIPTION OF DRAWINGS

FIG. 1 is a diagram of a test result of the expression of M2 and M1 macrophage markers when RAW264.7 cells were cultured in the presence of activin A or activin B.

FIG. 2 is a diagram of a test result of the expression of M2 macrophage markers when RAW264.7 cells were cultured in the presence of activin A.

FIG. 3 is a diagram of a test result of arginase mRNA expression in visceral fat when recombinant adenovirus was administered to db/db mice or high-fat diet-fed B6 mice to express activin A or activin B.

FIG. 4 is a diagram of a test result of the expression of M2 and M1 macrophage markers when RAW264.7 cells were cultured in the presence of activin A, activin B, or SB431542 with palmitic acid added.

FIG. 5 is a diagram of a test result of the expression of M2 and M1 macrophage markers when RAW264.7 cells were cultured in the presence of activin A, activin B, or Fstl3 with palmitic acid added.

FIG. 6 is a diagram of a test result of the expression of an M2 macrophage marker (arginase-1) when RAW264.7 cells were cultured in the presence of activin A, Fstl3, an anti-Fstl3 antibody, or mouse IgG with palmitic acid added.

FIG. 7 is a diagram of a test result of a glucose tolerance test (GTT) when recombinant adenovirus is administered to high-fat diet-fed B6 mice to express activin A or activin B.

### DESCRIPTION OF EMBODIMENTS

An active ingredient contained in an anti-inflammatory drug of the present invention is one or more species of activin selected from activin A, activin AB, or activin B. Activin A, activin AB, or activin B may be individually contained as the active ingredient or two or more species may be contained as the active ingredients. Activin species may be a protein derived from natural products, a protein produced by a genetic engineering technique, or a polypeptide (fragment) derived from such proteins on the condition that M2 macrophage-inducing activity exists. Activin A and activin B may be not only dimers but also sub-units making up dimers on the condition described above. A vector comprising a gene of activin species may also be available. Amino acids in the protein or polypeptide and nucleic acids in the gene may have one to several, for example, about one to five, additions, substitutions, or deletions. The protein produced by a genetic engineering technique can be used as a fusion with another protein or used after chemical modification by using a conventionally known technique for the purpose of enhancement of the M2 macrophage-inducing activity, storage stability and solubility as protein, etc.

Although the number of amino acids deleted, substituted, inserted, and/or added is one or more and no upper limit is particularly set, the number is that of amino acids that can be deleted, substituted, or added by a well-known method such as a site-specific mutagenesis method and is one to several dozen, preferably, 1 to 20, more preferably, 1 to 10, further preferably, 1 to 5.

To be a protein having activity as activin A, activin AB, or activin B or to be a polypeptide making up the protein having activity as activin A, activin AB, or activin B, a protein or a polypeptide contained in the anti-inflammatory drug of the present invention preferably has at least 60% or more, normally 80% or more, or 85% or more identity, more preferably 90% or more, particularly 95% or more identity with an amino acid sequence published as activin A, activin AB, or activin B by NCBI etc. It will easily be understood by those skilled in the art that a sequence of a gene of activin species making up a vector comprising the gene of activin species can be altered based on the description above.

The identity of amino acid sequence or base sequence can be determined by using the algorithm BLAST [Pro. Natl. Acad. Sci. USA, 90, 5873 (1993)] or FASTA [Methods Enzymol., 183, 63 (1990)] of Karlin and Altschul. Programs called BLASTN and BLASTX are developed based on the algorithm BLAST [J. Mol. Biol., 215, 403 (1990)]. If base sequence is analyzed by BLASTN based on BLAST, parameters are set to Score=100 and wordlength=12, for example. If amino acid sequence is analyzed by BLASTX based on BLAST, parameters are set to score=50 and wordlength=3, for example. If BLAST and Gapped BLAST programs are used, default parameters of the programs are used. Specific techniques of these analysis methods are known (http://www.ncbi.nim.nih.gov).

The term “induction of M2 macrophages” as used herein collectively refers to making M2 macrophages predominant in terms of quantitative or functional balance between M1 and M2 macrophages and means, for example, differentiation/induction from macrophage precursor cells to M2 macrophages, functional activation of M2 macrophages, and the enhancement of production of anti-inflammatory cytokines and enzymes such as IL-10 and arginase-1.

The term “inducer of M2 macrophages” as used herein refers to substances having any of the action or the effect. For example, the substances include activin species itself or a substance capable of neutralizing/suppressing a substance inhibiting the effect of activin species such as FSTL3, i.e., an anti-FSTL3 antibody, an expression inhibitor of FSTL3 gene such as antisense oligonucleotide, an FSTL3 mutant inhibiting binding between natural FSTL3 and activin, etc.

The origin of activin species is not limited on the condition that such activin species is acceptable for an animal when applied as an anti-inflammatory drug and, for example, activin species derived from any mammals such as human, monkey, pig, cattle, sheep, horse, and rat is usable.

When the anti-inflammatory drug of the present invention containing an activin species or an inducer of M2 macrophages as an active ingredient is used as a therapeutic drug, the drug can be included and used in a pharmaceutical composition. In such a case, any conventionally known carrier materials are usable. The carrier materials may be organic or inorganic carrier materials suitable for enteric, transdermal, or parenteral administration. Suitable carriers include water, gelatin, gum arabic, lactose, starch, magnesium stearate, talc, vegetal oil, polyalkylene glycol, vaseline, etc. A pharmaceutical preparation may contain another pharmaceutically active agent. Other additives such as flavoring agents, stabilizers, emulsifiers, and buffers may be added in accordance with the practice of pharmaceutical preparation.

A substance effectively suppressing inflammatory changes in adipocytes similar to the anti-inflammatory drug of the present invention can be screened by reference to the present description.

The anti-inflammatory drug of the present invention containing an activin species or inducer of M2 macrophages as an active ingredient and the substance effectively suppressing inflammatory changes in adipocytes similar to the anti-inflammatory drug of the present invention are used for human, monkey, pig, cattle, sheep, horse, rat, etc. In the case of human, the drug and the substance are particularly preferably used for individuals having, or potentially having, obesity, diabetes, or impaired glucose tolerance.

### EXAMPLES

### Example 1

(1) Induction of M2 Macrophages by Activin Species

Each of activin A (10 ng/mL or 30 ng/mL) and activin B (10 ng/mL or 30 ng/mL) was individually added to RAW264.7 cells. Human Recombinant (R&D Systems, 338AC005) and Recombinant (R&D Systems, 659AB005) were used for activin A and activin B, respectively. After eight hours, RNeasy Mini Kit (250) (Qiagen, Cat. Number 74106) was used for recovering the cells to extract RNA.

(2) Expression Analysis of Macrophage Markers

The RNA extracted at step (1) was used for performing a reverse transcription reaction in accordance with the protocol of High Capacity cDNA Reverse Transcription Kit with RNase Inhibitor (ABI) to perform TagMan PCR. Arginase-1 was selected as an M2 macrophage marker and IL-6 and MCP-1 were selected as M1 macrophage markers to create respective TaqMan probes. Each of mRNA expression levels is represented by a multiple when an expression level of cyclophilin A mRNA in the same sample is defined as one (hereinafter, the same applies to the examples using TaqMan PCR unless otherwise stated).

(3) Result

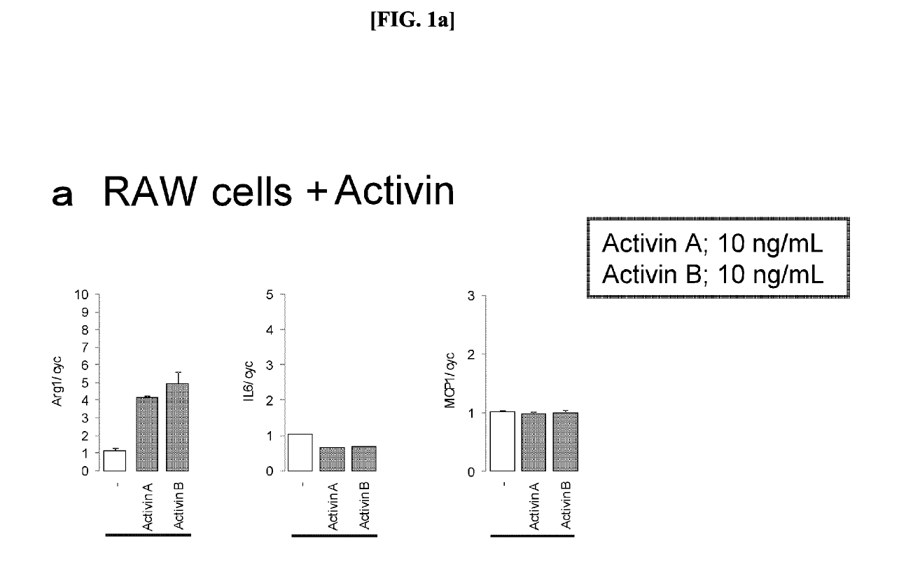

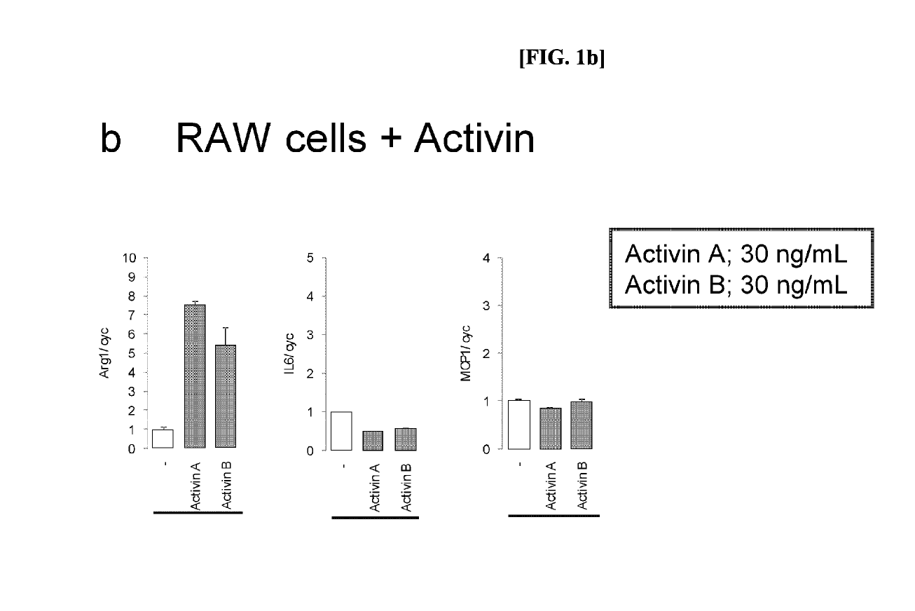

When activin A or activin B was individually added, the expression of arginase-1 was remarkably increased. On the other hand, the expression levels of IL-6 and MCP-1 were not changed. The expression level of arginase-1 was dependent on an additive concentration of activins. Therefore, it was confirmed that activin A and activin B induced M2 macrophages (see FIGS. 1a and 1b).

### Example 2

(1) Induction of M2 Macrophages by Activin Species

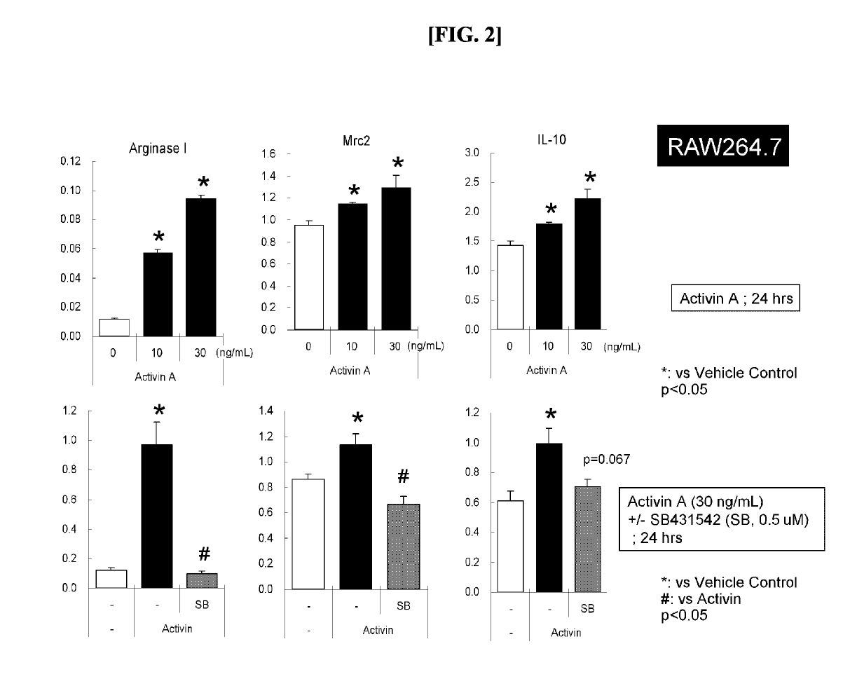

Activin A (10 ng/mL or 30 ng/mL) and SB431542 (0.1 to 1 μM, Santa Cruz Biotechnology, Inc., sc-204265) were added to RAW264.7 cells in combination as depicted in FIG. 2. Human Recombinant (R&D Systems, 338AC005) was used for activin A. After 24 hours, RNeasy Mini Kit (250) (Qiagen, Cat. Number 74106) was used for recovering the cells to extract RNA.

(2) Expression Analysis of Macrophage Markers

The RNA extracted at step (1) was used for performing a reverse transcription reaction in accordance with the protocol of High Capacity cDNA Reverse Transcription Kit with RNase Inhibitor (ABI) to perform TaqMan PCR. Arginase-1, Mrc2 (mannose receptor 2), and IL-10 were selected as M2 macrophage markers to create respective TaqMan probes.

(3) Result

When activin A was added, the expressions of arginase-1, Mrc2, and IL-10 were remarkably increased. This increase was reduced to the same level as control by the addition of an inhibitor SB431542 of activin receptors, activin-like kinase (ALK) 4/5/7. Therefore, the induction of M2 macrophages by activin A was further confirmed by the increases in the expression levels of Mrc2 and IL-10, which were M2 macrophage markers different from the first example (see FIG. 2).

### Example 3

(1) Production of Recombinant Adenovirus

Full-length mouse inhibin βA cDNA or full-length mouse inhibin βB cDNA was amplified by using pcDNA3.1/V5-HisA (Invitrogen) in accordance with the recommended method. The amplified product was subjected to HindIII and EcoRV treatments and used for production of recombinant adenovirus. The recombinant adenovirus of inhibin βA or inhibin βB was produced by using Takara Adenovirus Expression Vector Kit (Takara) in accordance with the recommended method. For negative control, β-galactosidase-gene-containing virus attached to the kit was used. The adenovirus was transfected to HEK293 cells by using CellPhect (registered trademark) Transfection Kit (GE Healthcare) with the calcium phosphate method.

(2) db/db mice

After seven-week-old male db/db mice were adapted for one week, 5.0×10¹¹ pfu/mL of the recombinant adenovirus was lysed in 150 μL of PBS and administered from the tail vein once a week for two weeks. Visceral fat was collected on seventh day after the second administration for performing a reverse transcription reaction in accordance with the protocol of High Capacity cDNA Reverse Transcription Kit with RNase Inhibitor (ABI) and the expression analysis of arginase-1 was performed with TaqMan PCR.

(3) Twelve-Week High-Fat Diet B6 Mice (High-Fat Diet-Fed Mice)

After five-week-old male B6 mice were adapted with normal diet for one week, high-fat diet was fed for twelve weeks. The recombinant adenovirus (5.0×10¹¹ pfu/mL) was lysed in 150 μL of PBS and administered to mice in the 18th week from the tail vein once a week for two weeks. Visceral fat was collected on seventh day after the second administration for performing reverse transcription in accordance with the protocol of High Capacity cDNA Reverse Transcription Kit with RNase Inhibitor (ABI) and the expression analysis of arginase-1 was performed with TaqMan PCR.

(4) Result

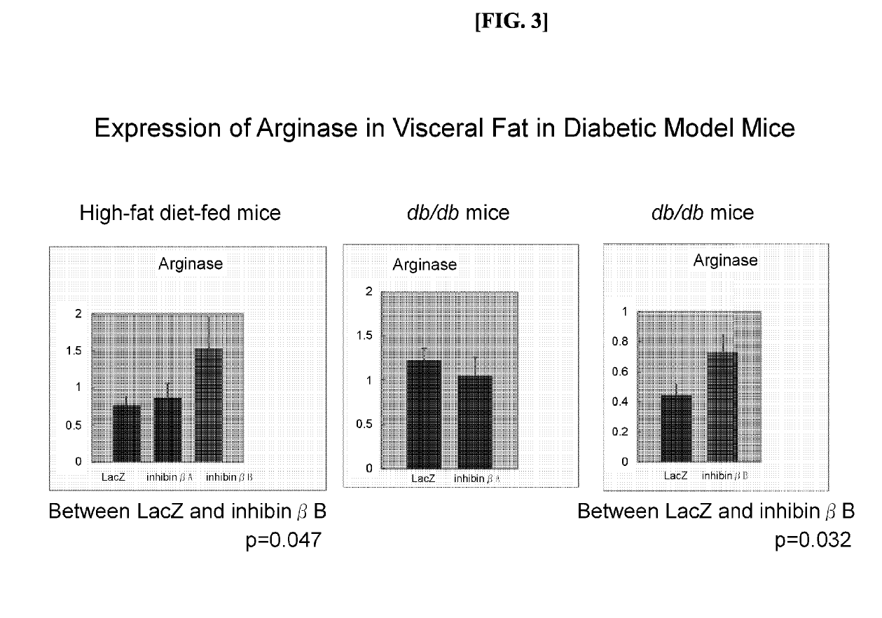

In the db/db mice, the expression of arginase-1 was remarkably increased by the expression of activin B (administration of inhibin βB recombinant adenovirus) as compared to the control. On the other hand, no difference from the control was recognized in the expression of arginase-1 due to the expression of activin A (administration of inhibin βA recombinant adenovirus). In the twelve-week high-fat diet B6 mice (high-fat diet-fed mice), the expression level of arginase-1 was similarly increased by the expression of activin B while the expression level of arginase-1 was also slightly increased when activin A was expressed (see FIG. 3).

As described in Example 2, when an inhibitor of activin receptors, SB431542, coexists, the arginase-1 expression level, remarkably increased by the overexpression of activin A, is reduced to the same level as the control. In light of this findings, it is conceivable that one of the reasons why no remarkable increase was found in the activin expression levels in visceral fat of genetically-obese db/db mice and high-fat diet-fed mice in this example is that the expression of FSTL3, inhibiting the binding of activins to the receptors, is also increased in the adipocytes of the model mice. Therefore, it is believed that desired induction of M2 macrophages can be achieved by adjusting the expression level of activin species with a method well-known to those skilled in the art.

### Example 4

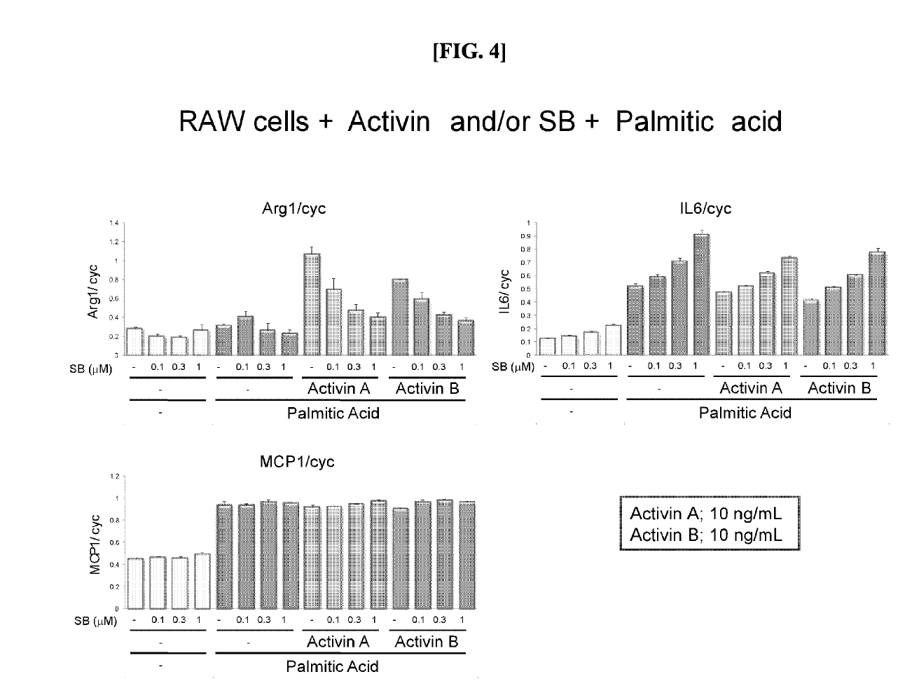

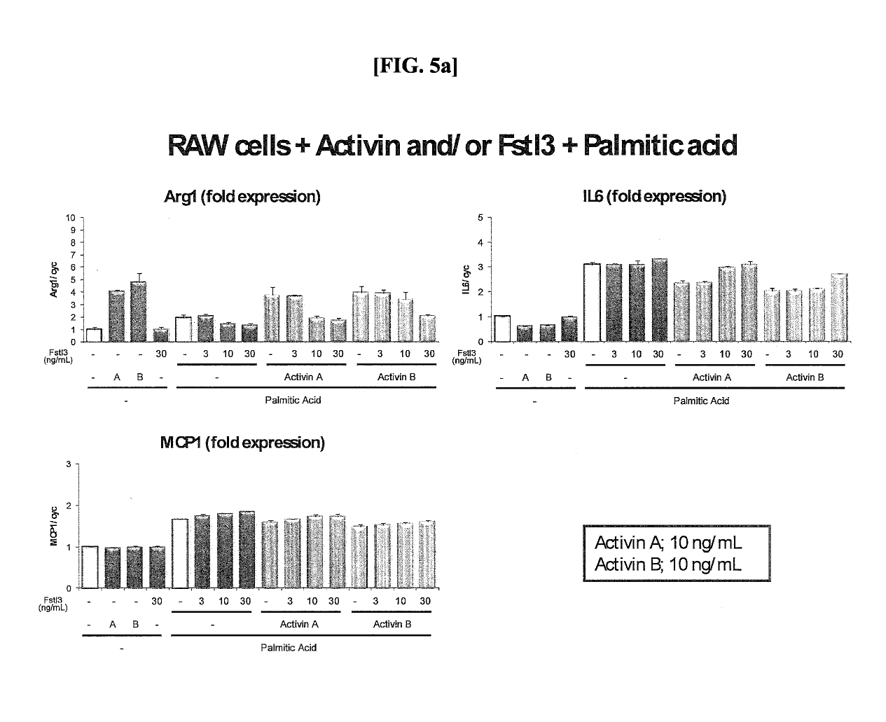

(1) Induction of Macrophages by Activin Species Activin A (10 ng/mL), activin B (10 ng/mL), and SB431542 (0.1 to 1 μM) were added to RAW264.7 cells in combination as depicted in FIG. 4. Human Recombinant (R&D Systems, 338AC005) and Recombinant (R&D Systems, 659AB005) were used for activin A and activin B, respectively. After 30 minutes, palmitic acid (a proinflammatory substance) was added to the final concentration of 200 μM and, after eight hours, RNeasy Mini Kit (250) (Qiagen, Cat. Number 74106) was used for recovering the cells to extract RNA.

(2) Expression Analysis of Macrophage Markers

The RNA extracted at step (1) was used for performing a reverse transcription reaction in accordance with the protocol of High Capacity cDNA Reverse Transcription Kit with RNase Inhibitor (ABI) to perform TaqMan PCR. Arginase-1 was selected as an M2 macrophage marker and IL-6 and MCP-1 were selected as M1 macrophage markers to create respective TaqMan probes.

(2) Expression Analysis of Macrophage Markers

Analysis was performed in the same way as the expression analysis of macrophage markers of Example 1.

(3) Result

(i) Addition of SB431542 Only (Background Control)

In this example, the case of absence of both the SB431542 and palmitic acid is simply referred to as control. When only the inhibitor SB431542 (0.1 to 1 μM) of the activin receptors, activin-like kinase (ALK) 4/5/7, was added without adding palmitic acid, the expression levels of arginase-1, IL-6, and MCP-1 were not changed or were slightly changed as compared to the control.

(ii) Addition of Palmitic Acid in the Absence of Activin Species

a) The expression level of arginase-1 was not changed or was slightly changed as compared to the control.

b) The expression level of IL-6 was remarkably increased by the addition of palmitic acid as compared to the control. The expression level was constantly increased depending on the additive concentration of SB431542.

c) The expression level of MCP-1 was remarkably increased by the addition of palmitic acid as compared to the control. This increase was not changed by the addition of SB431542.

(iii) Addition of Palmitic Acid in the Presence of Activin A

a) The expression level of arginase-1 was remarkably increased by the addition of palmitic acid as compared to the control. On the other hand, the expression level was reduced depending on the additive concentration of SB431542.

b) The expression level of IL-6 was remarkably increased by the addition of palmitic acid as compared to the control. The expression level was constantly increased depending on the additive concentration of SB431542. The expression of IL-6 was slightly reduced by the addition of activin A.

c) The expression level of MCP-1 was remarkably increased by the addition of palmitic acid as compared to the control. This increase was not changed by the addition of SB431542. The expression level of MCP-1 was not changed by the addition of activin A.

(iv) Addition of Palmitic Acid in the Presence of Activin B

a) The expression level of arginase-1 was remarkably increased by the addition of palmitic acid as compared to the control. On the other hand, the expression level was reduced depending on the additive concentration of SB431542.

b) The expression level of IL-6 was remarkably increased by the addition of palmitic acid as compared to the control. The expression level was constantly increased depending on the additive concentration of SB431542. The expression of IL-6 was slightly reduced by the addition of activin B.

c) The expression level of MCP-1 was remarkably increased by the addition of palmitic acid as compared to the control. This increase was not changed by the addition of SB431542. The expression level of MCP-1 was not changed by the addition of activin B.

(v) Conclusion

Although the expression levels of the M1 macrophage markers (IL-6 and MCP-1) were increased by the addition of palmitic acid that is a proinflammatory substance, the expression level of the M2 macrophage marker (arginase-1) was not changed. This result conformed to the previously reported behaviors of M1 and M2 macrophages in inflammation. On the other hand, when activin A or activin B was present together, the expression level of arginase-1 was enhanced even when palmitic acid was added. This is believed to indicate that when macrophages are stimulated, activin species have an effect of inducing (activating) M2 macrophages. The expression level of IL-6 was relatively lower when an activin species was present together as compared to the case that an activin species was not present together. This is believed to indicate that activin species have an effect of suppressing the IL-6 production in M1 macrophages. As a result, it is found out that activin species can be used as an anti-inflammatory drug producing an anti-inflammatory effect by modulating macrophage function (see FIG. 4).

### Example 5

(1) Induction of Macrophages by Activin Species

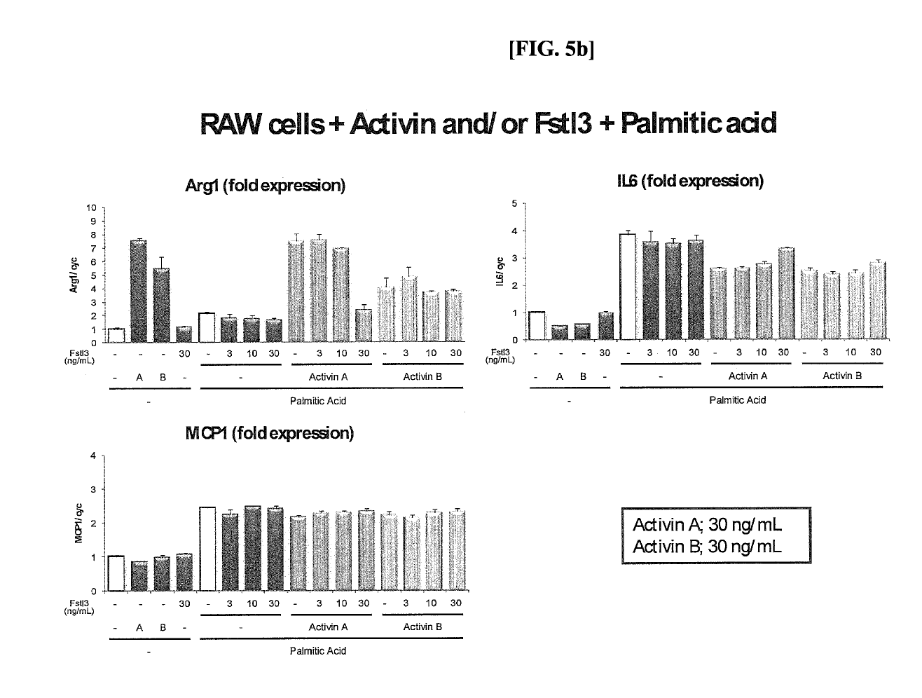

Activin A (10 ng/mL), activin B (10 ng/mL), and Fstl3 (3 to 30 ng/mL) were added to RAW264.7 cells in combination as depicted in FIG. 5. Human Recombinant (R&D Systems, 338AC005), Recombinant (R&D Systems, 659AB005), and Follistatin-like 3 Recombinant (R&D Systems, 1255F3025) were used for activin A, activin B, and Fstl3, respectively. After 30 minutes, palmitic acid (a proinflammatory substance) was added to the final concentration of 200 μM and, after eight hours, RNeasy Mini Kit (250) (Qiagen, Cat. Number 74106) was used for recovering the cells to extract RNA.

(2) Expression Analysis of Macrophage Markers

Analysis was performed in the same way as Example 4. For each of palmitic acid, activin species, and Fstl3, the expression level is represented by a ratio when a measurement value of each marker relative to a measurement value of cyclophilin A in non-additive control is defined as one.

(3) Result

Even in a cultured cell system having inflammation caused by palmitic acid, the expression of arginase-1 was induced by the addition of activin species (an activin species added without adding Fstl3 in the presence of palmitic acid). When Fstl3 was added to culture solution concurrently with activin A, the expression of arginase-1 was reduced with the additive amount of Fstl3 as 30 ng/mL. It is believed that this is because the binding of Fstl3 to activin A inhibits the M2 macrophages-inducing effect of activin A. This has also led to an inference that as Fstl3, sufficient for inhibiting the effect of activin A, was expressed in the db/db mice of Example 3, the expression of arginase-1 was not increased.

The expression of the M1 macrophage marker, IL-6, was increased by the addition of palmitic acid and reduced by the addition of activins. The addition of activin species induced M2 macrophages, which produces anti-inflammatory cytokines, and reduced the expression of inflammatory cytokines, which indicates the anti-inflammatory effect of activin species.

On the other hand, although the expression of MCP1 was increased by the stimulation of palmitic acid regardless of the presence of activin species, the change was negligible and the expression level was not changed by the presence of FSTL3. It is inferred that the expression of MCP1 in inflammation induced by palmitic acid is controlled through a pathway different from the signal transduction via activin species (see FIGS. 5a and 5b).

### Example 6

(1) Induction of M2 Macrophages by Inducer of M2 Macrophages

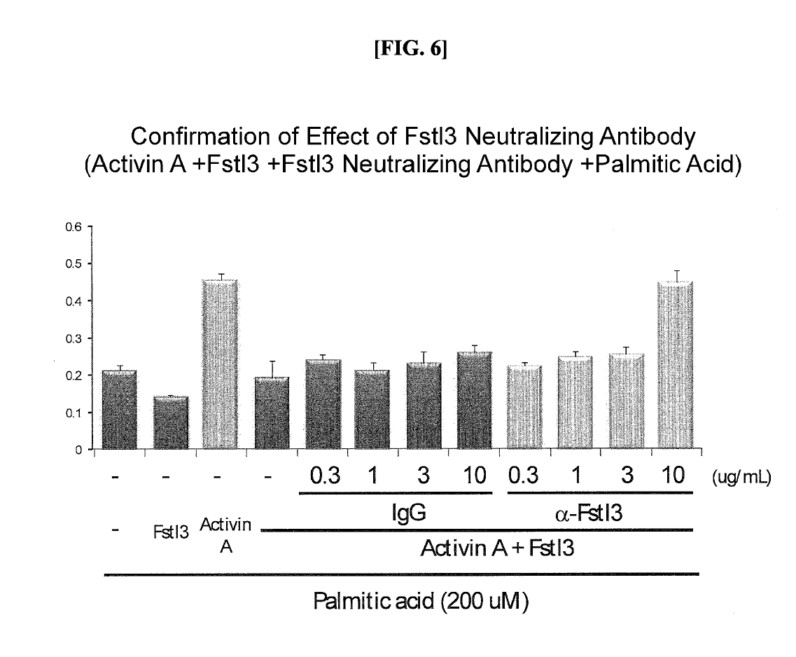

Fstl3 (30 ng/mL), an anti-Fstl3 antibody or mouse IgG (0.3 to 10 μg/mL), and activin A (10 ng/mL) were added to RAW264.7 cells in combination as depicted in FIG. 6. Human Recombinant (R&D Systems, 338AC005), Recombinant (R&D Systems, 659AB005), and Follistatin-like 3 Recombinant (R&D Systems, 1255F3025) were used for activin A, activin B, and Fstl3, respectively. After 30 minutes, palmitic acid (a proinflammatory substance) was added to the final concentration of 200 μM and, after eight hours, RNeasy Mini Kit (250) (Qiagen, Cat. Number 74106) was used for recovering the cells to extract RNA.

(2) Expression Analysis of Macrophage Markers

The RNA extracted at step (1) was used for performing a reverse transcription reaction in accordance with the protocol of High Capacity cDNA Reverse Transcription Kit with RNase Inhibitor (ABI) to perform TaqMan PCR. Arginase-1 was selected as an M2 macrophage marker to create TaqMan probes.

(3) Result

Although the expression level of arginase-1 was increased by the addition of activin A even under the induction of inflammation by palmitic acid, when FSTL3 was present at the same time, the expression level of arginase-1 was reduced to the same level as the control without activin A and FSTL3.

Although the expression level of arginase-1 was not changed by the addition of mouse IgG, when 10 ng/mL of the anti-FSTL3 antibody was added, the expression amount of arginase-1 was recovered to the same level as when FSTL3 was not added. Under the condition without the stimulation of palmitic acid, the expression level of arginase-1 was recovered by a lower concentration of the anti-FSTL3 antibody (data not shown) and, therefore, an optimum anti-inflammatory effect can be acquired by adjusting the amount of inducer of M2 macrophages as needed depending on the level of inflammation (see FIG. 6).

### Example 7

(1) Production of Recombinant Adenovirus

Adenovirus was produced in the same way as the production of recombinant adenovirus in Example 3.

(2) Twelve-Week High-Fat Diet B6 Mice (High-Fat Diet-Fed Mice)

After five-week-old male B6 mice were adapted with normal diet for one week, high-fat diet was fed for twelve weeks. The recombinant adenovirus (5.0×10¹¹ pfu/mL) was lysed in 150 μL of PBS and administered to mice in the 18th week from the tail vein once a week for two weeks. A glucose tolerance test (GTT) was performed on seventh day after the second administration.

(3) Glucose Tolerance Test (OTT)

The GTT was performed as follows. Into the abdominal cavities of mice fasted for 16 hours from the previous day of the test, 1.0 mg/g-BW of glucose (diluted by physiological saline) was administered. Serum was separated from collected whole blood and glucose concentration was measured by Glucose-Test Wako (manufactured by Wako Pure Chemical Industries).

(4) Result

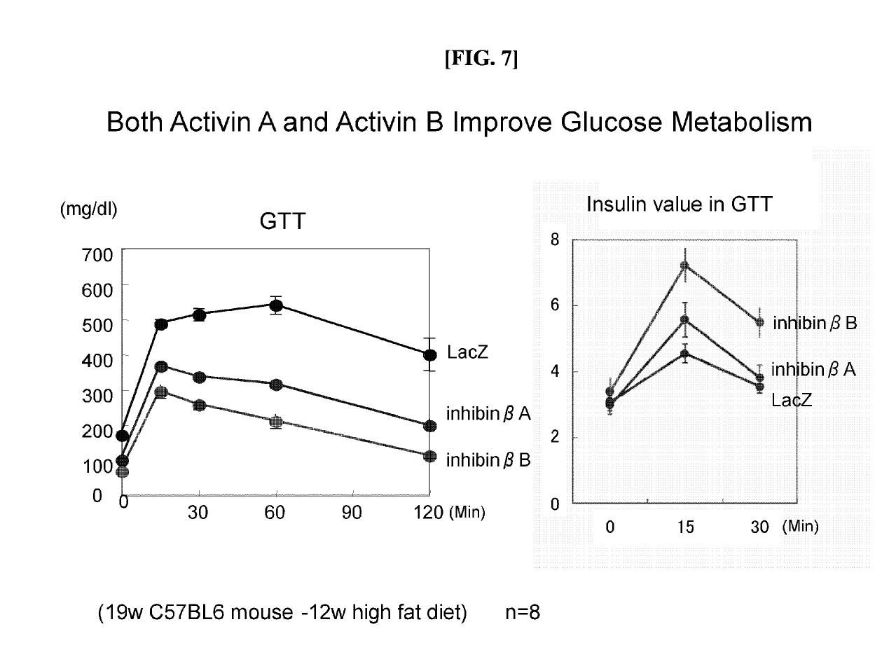

When activin A or activin B was expressed in the mice exhibiting a symptom of diabetes (insulin resistance) due to high-fat diet, both activin A and activin B improved glucose metabolism. It is found out that the modulation of macrophage function by activin species actually leads to the improvement of clinical conditions (see FIG. 7).

1. An anti-inflammatory drug comprising an activin species as an active ingredient.

2. The anti-inflammatory drug according to claim 1, wherein said activin species is one or more species selected from activin A, activin AB, or activin B.

3. The anti-inflammatory drug according to claim 1, wherein said activin species is a vector in which a gene of said activin species is incorporated.

4. An anti-inflammatory drug comprising an inducer of M2 macrophages as an active ingredient.

5. A method of treating or improving inflammation comprising: a step of administering an activin species to a subject.

6. The anti-inflammatory drug according to claim 2, wherein said activin species is a vector in which a gene of said activin species is incorporated.
